(19) United States
(12) Patent Application Publication
(10) Pub. No.: US 2017/0348277 A1
(43) Pub. Date: Dec. 7, 2017

(54) CANNABINOID FORMULATION INCLUDING A VASODILATOR AND OCULAR DELIVERY OF THE SAME

(71) Applicant: Bryan Ray Ruiz, Visalia, CA (US)

(72) Inventor: Bryan Ray Ruiz, Visalia, CA (US)

(21) Appl. No.: 15/667,557

(22) Filed: Aug. 2, 2017

(51) Int. Cl. A61K 31/352; A61K 9/127; C07C 317/04; C07D 311/78; A61K 9/00

(52) U.S. Cl. CPC A61K 31/352; A61K 9/127; C07C 317/04; A61K 9/0043; A61K 9/0048; C07D 311/78

(57) ABSTRACT

A cannabinoid formulation and method for ocular or nasal delivery. The method of administration includes providing eye drops that comprise an aqueous solution of at least one water soluble cannabinoid, saline, and a vasodilator; and administering the eye drops topically to an eye of the subject. In an alternate method the cannabinoid formulation is a nasal spray delivered through the nose of a subject. Preferably, the vasodilator is 0.9% methylsulfonylmethane (MSM) in a MSM solution so that the MSM solution has a concentration of 5-15% in the overall formulation. Additionally, the at least one water soluble cannabinoid is encapsulated with a phospholipid liposome.

### FIELD OF THE INVENTION

The present invention relates to cannabinoid formulations. More particularly, the present invention relates the use of a vasodilator to improve the ocular and nasal delivery of cannabinoids in vivo.

### BACKGROUND OF THE INVENTION

A cannabinoid is a chemical compound that acts on cannabinoid receptors in cells within the body of a subject. Cannabinoids alter neurotransmitter release in the brain. There are three categories of cannabinoids. These include endocannabinoids, phytocannabinoids, and synthetic cannabinoids. Endocannabinoids are produced within the bodies of animals including humans. Phytocannabinoids are produced by plants. Synthetic cannabinoids are typically manufactured in a lab environment.

At least 113 different phytocannabinoids have been isolated from the Cannabis Sativa plant. Cannabis Sativa includes numerous varieties of hemp, including industrial hemp. Cannabis Sativa also includes numerous species of marijuana including sativa, indica and ruderalis. Each species has a multitude of phenotypes.

Classical cannabinoids are the most widely known and available in the Cannabis Sativa plant. The most notable of classical cannabinoids are Tetrahydrocannabinol (THC), Cannabidiol (CBD), and Cannabigerol (CBG). These classical cannabinoids are widely known for their health-enhancing and therapeutic properties. The molecules are defined below. It can be appreciated that isomers of these molecules are generically encompassed by the named molecules.

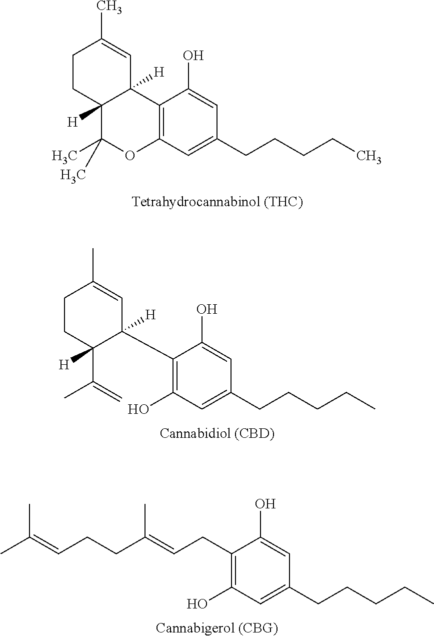

There are various cannabinoids receptors in animals including humans, dogs, and cats. The cannabinoid receptor type 1 (CB1 receptor) is typically found in the brain and other areas of the body. The CB1 receptors are also found in anterior eye and in the retina.

The cannabinoid receptor type II (CB2 receptor) is predominantly found in the immune system with the greatest density in the spleen Immune function and inflammatory responses are affected by the activity of the CB2 receptors.

While numerous ways of formulating cannabinoids for administration to the subject have been devised, there's still are challenges with the effective delivery of cannabinoids. For example, orally consumable cannabinoids typically degrade in the digestive tract and some have determined that less than 5% of the orally consumed cannabinoids actually reach the bloodstream. Further there is a 1 to 2 hour latency period associated with orally consumed cannabinoids. Orally consumable in this context means taken by mouth in the form of a pill, oil, lozenge, or in the form of an edible product.

Smoking the flowers of cannabis sativa is effective, but this delivery method is disfavored by the ill, infirm, and those that prefer not to ingest smoke having possibly carcinogenic components.

Vaporizing the oil of cannabis sativa is also effective, but many (including animals) are not able to operate a vaporization device. Others associate vaporizing with smoking and still others lack the equipment to accomplish vaporizing cannabis oil. Some believe that some bioactive and therapeutic components are lost in the vaporizing process. Those in the midst of a seizure would have difficulty with administration of cannabinoids with a vaporizer.

What is desired is a way of ingesting cannabinoids that is optimally effective and easy to accomplish. What is also desired is a way of ingesting cannabinoids for the infirm. What is also desired is a way of ingesting cannabinoids that is effective at remedying numerous diseases. Further it is desired to have a way of delivering cannabinoids to animals including cats and dogs.

### SUMMARY OF THE INVENTION

A cannabis formulation including a vasodilator is packaged in an eye dropper or nasal sprayer. In one embodiment, the formulation includes an aqueous solution of at least one water soluble cannabinoid, saline in the form of saline solution mixed with the aqueous solution, and a vasodilator. Preferably, the formulation is particularly designed to rapidly and effectively agonize CB1 receptors through ocular or nasal delivery.

In a preferred embodiment, the vasodilator is methylsulfonylmethane (MSM), which is also known as dimethyl sulfone (DMSO2) and methyl sulfone. Studies have shown that MSM is safe in doses of up to 2 gm/kg of body weight per day when taken orally. Since the formulation is delivered through the eye in the presence of the vasodilator the amount of cannabinoids from the cannabinoid formulation is maximized. The enables the concentration of the water soluble cannabinoid is less than 0.1 mg/ml. In one embodiment the concentration is between 0.01 and 0.1 mg/ml. In another embodiment, the at least one water soluble cannabinoid is encapsulated with a liposome.

The cooperation of MSM and the at least one cannabinoid causes the at least one cannabinoid to achieve a sufficient degree of water solubility to function. Thus the MSM has two functions, the first causing a lipophilic cannabinoid to become adequately water soluble, and the second is to increase blood flow at the point of deliver to optimize deliver of the cannabinoid through the vasculature of a subject. Further nasal and ocular delivery of the cannabinoid to the blood stream ports the cannabinoid directly into the brain. When combined with the vasodilator, this occurs quickly. In one embodiment, the formulation is administered to a subject in the midst of a seizure and stops the seizure.

In one embodiment, the vasodilator such as MSM is 5-15% of the formulation. In a 30 ml batch, the MSM solution added is between 1-6 ml. Preferably between 4-5 ml of MSM is used. More preferably 4.5 ml is used. It can be appreciated that greater or lesser amounts may be added and remain safe for the subject.

The cooperation of the benefit of the chosen vasodilator with the liposome encapsulated cannabinoids enables not only rapid delivery, but also rapid bioavailability through the cell walls of a subject to optimally achieve desired therapeutic effects.

In various embodiments, the present invention is used in conjunction with veterinary medicine. Thus canine, feline, bovine, swine, and equestrian administration of the present invention is anticipated.

### DESCRIPTION OF THE DRAWING

FIG. 1 is a liposome.

### DETAILED DESCRIPTION

The present invention is particularly useful for treating cataracts and other eye conditions including infections, myopia, hyperopia, eyestrain, retinal detachment, tumors, and glaucoma. These conditions are examples, and many other conditions can be treated in accordance with the present invention including diabetes, and asthma when delivered nasally

The present invention is also particularly useful for treating neurological conditions including seizures, and locked-in-syndrome. These are just two of many possibilities of neurological conditions that can be treated.

In an embodiment of the invention, the formulation is administered as eye drops to an animal such as livestock, or a pet such as a dog or cat. 1-5 eye drops are administered.

Accordingly, the present invention includes methods of treatment of the diseases disclosed herein as well as other diseases that have been remedied by the use of cannabinoids. The present invention is particularly useful for treating diseases of the eye, and for delivering cannabinoids into the optic nerve, the brain, and the vasculature of a subject. This enables many treatment possibilities, particularly for those suffering with glioblastoma. Since the present invention delivers cannabinoids rapidly and directly to the vasculature of the brain, the present invention is also optimal for treatment of seizures as they occur. In one embodiment, the present invention is delivered in the form of a nasal spray to administer to a subject when the subject is having a seizure.

Phytocannabinoids employed by the present invention preferably include any combination of the following classical cannabinoids derived from Cannabis Sativa:
THC (Tetrahydrocannabinol)
THCA (Tetrahydrocannabinolic acid)
CBD (Cannabidiol)
CBDA (Cannabidiolic Acid)
CBN (Cannabinol)
CBG (Cannabigerol)
CBC (Cannabichromene)
CBL (Cannabicyclol)
CBV (Cannabivarin)
THCV (Tetrahydrocannabivarin)
CBDV (Cannabidivarin)
CBCV (Cannabichromevarin)
CBGV (Cannabigerovarin)
CBGM (Cannabigerol Monomethyl Ether)
CBE (Cannabielsoin)
CBT (Cannabicitran)

In one embodiment, the present invention includes solely acid forms of cannabinoids to minimize psychoactive effects associated with THC. In another embodiment, the cannabinoids are solely CBD to minimize psychoactive effects. In another embodiment, a combination of THC-A and CBD are formulated together. It can be appreciated that any combination of the classical cannabinoids and other cannabinoids can be used in accordance with the present invention.

While the present invention describes the use of isolated cannabinoids, preferably having an 80-99.9% purity, the present invention can also be optimized using a whole plant extract having a cannabinoid profile roughly paralleling the cannabinoid profile in a substrate plant material.

When using isolated cannabinoids, isolated cannabinoids are formulated to be water soluble with sufficient water solubility to remain suspended in an aqueous solution for a desired shelf life, e.g. two years.

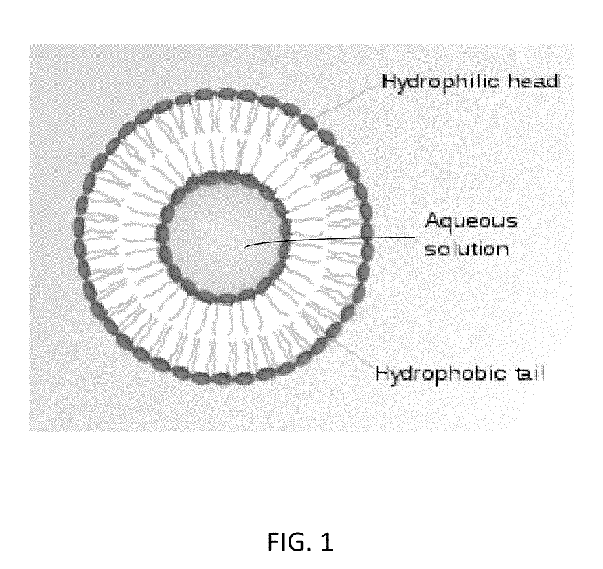

FIG. 1 shows one embodiment wherein the cannabinoids are encapsulated with nano-sized (less than 1 micron) liposomes. The liposomes define a core housing cannabinoids in an aqueous solution. The cannabinoids are adapted to be sufficiently soluble to achieve suspension in an aqueous solution that is encapsulated by liposomes.

In one embodiment the liposome encapsulated cannabinoid (e.g. CBD) includes an isolated cannabinoid core surrounded by a hydrophobic membrane. This yields a lipid bilayer. The cannabinoid in the core does not readily pass through the bilayer. Since the cannabinoids are typically hydrophobic, they associate with the bilayer.

In delivering the cannabinoids to a site of action, the lipid bilayer can interact with other bilayers such as the cell membrane of a vascular cell, and more easily enter into the blood stream. This is particular useful when combined in cooperation with a vasodilator that improves the flow of blood in the region of delivery.

In an alternate embodiment, the vasodilator is encapsulated by the liposome bilayer using an appropriate liposome, and is delivered simultaneously in a mixture with liposome encapsulated cannabinoids. In another embodiment, the vasodilator and the cannabinoid are both encapsulated by the same liposome bilayer. It can be appreciated that although a liposome bilayer is utilized by the present invention, a liposome monolayer can be employed with lipophilic cannabinoids including THC and CBD.

Thus it is possible not only to deliver the liposome core contents to the blood stream, the liposome layer(s) can be designed to optimally deliver the liposome core contents directly to the neurons. This is especially effective where ocular delivery is utilized.

In an alternate embodiment, the liposomes used are designed to deliver cannabinoids via alternate pathways such as diffusion. WE speculate that liposomes that contain low (or high) pH can be constructed to use electrical charge to further optimize delivery to the blood stream, or to a particular site of action i.e. the brain. As the pH naturally neutralizes within the liposome (protons can pass through some membranes), the charged cannabinoids will also be neutralized, allowing it to freely pass through a membrane. These liposomes work to deliver drug by diffusion rather than by direct cell fusion.

Preferably, a raw phospholipid liposome used in the formulation is derived from lecithin. Phosphatidylcholine is a specific example of a liposome that is useful for the purposes described herein. The formulation can be adapted to alternate liposomes to create homogeneous liposome particle sizes that are stable and hold their encapsulated payload of cannabinoids in the core for a pre-determined period, or until they reach the most desirable point of delivery.

Encapsulating the cannabinoids, vasodilators or other molecules used herein i.e. MSM, is accomplished by supplying energy to a dispersion of phospholipids in a polar solvent, such as water, to break down multilamellar aggregates into oligolamellar bilayer vesicles. The vasodilator is an oxide of sulphur, having two double-bonded oxygen molecules.

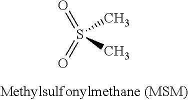

Liposomes can hence be created by sonicating a dispersion of amphipatic lipids, such as phospholipids, in water. Sonication is one method of preparation. Extrusion and the Mozafari method may also be employed to produce liposomes for encapsulating cannabinoids. In one embodiment phosphatidylcholine is used. It can be appreciated that other lipids can be used liposome preparation.

Various vasodilators have been studied that have the desired effect of increasing the rate of delivery of cannabinoids to a desired site of action. While the following classes of vasodilators may be utilized with the present invention, MSM has proven to be sufficiently effective, and also functions as a preservative to improve shelf life. The classes of vasodilators that may be substituted for the MSM of the present invention either in a preferred combination or in isolation. These classes include:
Alpha-adrenoceptor antagonists (alpha-blockers)
Angiotensin converting enzyme (ACE) inhibitors
Angiotensin receptor blockers (ARBs)
Beta₂-adrenoceptor agonists (β₂-agonists)
Calcium-channel blockers (CCBs)
Centrally acting sympatholytics
Direct acting vasodilators
Endothelin receptor antagonists
Ganglionic blockers
Nitrodilators
Phosphodiesterase inhibitors
Potassium-channel openers
Renin inhibitors

Various examples of preferred formulation manufacturing methods and formulation components are provided as follows:

### Example Formulation Method #1

Producing a 30 ml batch with:
10 ml purified water;
2.5 mg of isolated THC in a water-soluble form.
2 ml Opti-MSM® methylsulfonylmethane 0.9% solution
20 ml of 0.9% saline solution
Mixing and heating the mixture at 210° F. for one hour in a flask with a magnetic stirrer.
Filtering with a 5 micron filter
Packaging in five 6 ml glass eyedropper bottles.

### Example Formulation Method #2

Producing a 30 ml batch with:
8 ml purified water;
2.5 mg of liposome encapsulated CBD (the CBD having 99% purity)
2 ml Opti-MSM® methylsulfonylmethane 0.9% solution
20 ml of 0.9% saline solution
Mixing and heating the mixture at 210° F. for one hour in a flask with a magnetic stirrer.
Filtering with a 5 micron filter
Packaging in a glass eyedropper bottle.

### Example Formulation Method #3

Producing a 30 ml batch with:
10 ml purified water;
0.5 mg of liposome encapsulated CBD (80% purity powder)
4 ml methylsulfonylmethane 0.9% solution
16 ml of 0.9% saline solution
Mixing and heating the mixture at 210° F. for one hour in a flask with a magnetic stirrer.
Filtering with a 5 micron filter
Packaging in a glass eyedropper bottle.

### Example Formulation Method #4

Producing a 30 ml batch with:
10 ml purified water;
0.5 mg of liposome encapsulated CBG
2 ml methylsulfonylmethane 0.9% solution
18 ml of 0.9% saline solution
Heating the mixture at 210° F. for one hour in a flask.
Stirring with a magnetic stirrer for three hours.
Filtering with a 5 micron filter
Packaging in a nasal spray dispenser.

### Example Formulation Method #5

Producing a 30 ml batch with:
10 ml purified water;
2.5 mg of whole plant extract of cannabinoids (80% cannabinoid content)
4.5 ml Opti-MSM® Methylsulfonylmethane 0.9% solution
18 ml of 0.9% saline solution
Heating the mixture at 210° F. for one hour in a flask.
Stirring with a magnetic stirrer for three hours.
Filtering with a 5 micron filter
Packaging in an eye drop dispenser.

The present invention envisions transmucosal administration of the present invention via the ocular or nasal routes. For ocular delivery, the number of drops is between (1-5). For nasal delivery, the same volume in the form of a mist is administered. The nature of administration and formulation reduces the need for administering a large volume of cannabinoids. To compare, an orally administered therapeutic dose of THC, for example, via an edible product can be 10-25 mg. In accordance with the present invention, 0.1 mg or less can be therapeutic. Subjective testing indicates that the present invention may be more than 100× more potent than traditional oral deliver via an edible product.

The product and methods of the present invention also can be employed through delivery via the ears, sublingually and topically.

I claim:

1. Formulated cannabinoid eye drops comprising:
an aqueous solution of at least one water soluble cannabinoid;
saline, and
a vasodilator.

2. The formulated cannabinoid eye drops of claim 1, wherein the vasodilator is methylsulfonylmethane (MSM), defined by the formula:

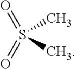

3. The formulated cannabinoid eye drops of claim 1, wherein the vasodilator is an oxide of sulphur.

4. The formulated cannabinoid eye drops of claim 1, wherein the vasodilator is methylsulfonylmethane (MSM) and the concentration of the at least one water soluble cannabinoid is less than 0.1 mg/ml.

5. The formulated cannabinoid eye drops of claim 1, wherein the vasodilator is methylsulfonylmethane (MSM), the concentration of the water soluble cannabinoid is less than 0.1 mg/ml, and the at least one water soluble cannabinoid is encapsulated with a liposome.

6. The formulated cannabinoid eye drops of claim 1, wherein the vasodilator is Methylsulfonylmethane (MSM), and the at least one water soluble cannabinoid is encapsulated with a phospholipid liposome.

7. A method of treating a subject comprising:
providing eye drops comprising an aqueous solution of at least one water soluble cannabinoid, saline, and a vasodilator;
administering the eye drops topically to an eye of the subject.

8. The method of claim 7, wherein the vasodilator is methylsulfonylmethane (MSM).

9. The method of claim 7, wherein the vasodilator is includes a compound including sulphur.

10. The method of claim 7, wherein the vasodilator is methylsulfonylmethane (MSM) and the concentration of the at least one water soluble cannabinoid is less than 0.1 mg/ml.

11. The method of claim 7, wherein the vasodilator is methylsulfonylmethane (MSM), the concentration of the water soluble cannabinoid is less than 0.1 mg/ml, and the at least one water soluble cannabinoid is encapsulated with a liposome.

12. The method of claim 7, wherein the vasodilator is methylsulfonylmethane (MSM), and the at least one water soluble cannabinoid is encapsulated with a phospholipid liposome.

13. The method of claim 7, wherein the method treats cataracts.

14. The method of claim 7, wherein the method treats glioblastoma.

15. The method of claim 7, wherein the method treats locked-in-syndrome.

16. The method of claim 7, wherein the subject it a canine.

17. The method of claim 7, wherein the subject it a feline.

18. The method of claim 7, wherein the method treats a seizure.

19. The method of claim 7, wherein the method treats myopia.

20. The method of claim 7, wherein the method treats eye strain.
